(19) United States
(12) Patent Application Publication
(10) Pub. No.: US 2011/0008912 A1
(43) Pub. Date: Jan. 13, 2011

(54) Polymer-coated substrates for binding biomolecules and methods of making and using Thereof

(76) Inventors: Anthony G. Frutos, Painted Post, NY (US); Joydeep Lahiri, Painted Post, NY (US); Jinlin Peng, Painted Post, NY (US); Odessa N. Petzold, Elmira, NY (US)

Correspondence Address: CORNING INCORPORATED, SP-TI-3-1, CORNING, NY, 14831, US

(21) Appl. No.: 12/886,997

(22) Filed: Sep. 21, 2010

Related U.S. Application Data
(62) Division of application No. 10/996,952, filed on Nov. 24, 2004, now abandoned.

(51) Int. Cl. G01N 33/53; C08F 20/08

(52) U.S. Cl. 436/501; 525/327.4; 525/54.1; 525/54.2

(57) ABSTRACT

Described herein are polymer-coated substrates for binding biomolecules and methods of making and using thereof.

### BACKGROUND

Assays using label independent detection (LID) platforms (e.g. surface plasmon resonance (SPR) or resonant grating sensors) are typically performed using a two step procedure: (i) immobilization of one of the binding partners (typically a protein) on the surface of the sensor; and (ii) binding of a ligand (drug, protein, oligonucleotide, etc) to the immobilized protein. Traditionally, the coupling of biomolecules to surfaces involves the activation of carboxylic acid groups on the surface to reactive N-hydroxysuccinimide (NHS) esters, which are then coupled to amino groups on the protein of interest. This method has been successfully used and commercialized by Biacore, Affinity Biosensors, and Artificial Sensing Instruments for their respective LID platforms. While effective, the activation step is time consuming and involves the handling and use of somewhat toxic chemicals.

An alternative to this approach involves the use of “preactivated” chemistries. For example, surfaces presenting aldehyde groups have been used to bind biomolecules. However, a reduction step is required after coupling to stabilize the resulting Schiff base. Surfaces with epoxide and isocyanate functionalities have also been used; however, the epoxide group is relatively slow to react and, therefore, requires long incubation times under very basic conditions, while the isocyanate group is extremely reactive and presents storage stability issues. Because of these issues, there are few reports of the use of preactivated chemistries for LID platforms. In fact, neither Biacore, Affinity Biosensors, nor ASI—the three companies offering the most popular LID platforms—offer sensors with a preactivated chemistry.

Maleic anhydride reacts readily with nucleophiles such as amino groups. Although the modification of surfaces with maleic anhydride copolymer layers for the immobilization of small molecules, DNA, sugars, and peptides has been described (1-9), the hydrolytic stability of maleic anhydrides is rather poor (10), and for this reason they have not been widely used. The hydrolytic stability of maleic anhydride can be increased when copolymerized with hydrophobic side chains (e.g. styrene); however, this leads to problems with nonspecific binding of biomolecules to the surface. While this may be an advantage for some applications such as mass spectrometry, it is problematic for LID.

There is a unique issue with LID detection in general that necessitates a stringent requirement for biospecificity. The incorporation of “blocking agents” (e.g. bovine serum albumin, BSA) in the analyte solution is undesirable because both specific (due to the analyte) and non-specific (due to the blocking agent) binding would contribute to changes in interfacial refractive index and would hence be indistinguishable. This problem is only exacerbated when complex samples are used or when the analyte is impure. The concern with anhydrides for immobilization of proteins is non-specific binding due to the formation of residual negative charge and the influence of other groups (e.g. styrene, ethylene, methyl vinyl ether, etc) in the polymer. Because of these reasons, the feasibility of using anhydride polymers for LID is a potential concern. This concern, coupled with potential stability issues of the anhydride group is one reason why the use of anhydride copolymers for LID applications is not currently known or described in the prior art. This invention describes how maleic anhydride polymers can be successfully used in LID assays. By the appropriate selection of the side chain in the polymer and immobilization conditions, binding assays can be performed with high specificity while maintaining sufficient hydrolytic stability. Moreover, this invention discloses that immobilization of many biomolecules on maleic anhydride copolymer surfaces can be accomplished under acidic (pH<7) conditions with the advantages of increased hydrolytic stability and increased amount of protein binding relative to more traditional peptide coupling conditions (pH 7-9).

Immobilization using a 3D matrix enables a greater amount of immobilization of the biomolecule and hence a greater number of sites for binding. Hydrogels such as carboxymethyldextran are the most common (32-34). A concern with hydrogels is the partitioning of large analyte molecules to binding sites within the hydrogel proximal to the surface. Relative to conventional detection by techniques such as fluorescence microscopy, there is rapid decay of the binding signal away from the surface because of the exponential nature of the evanescent electromagnetic field for LID detection. The polymeric surfaces described herein are not as thick as hydrogels and, thus, immobilization occurs closer to the interface, which can circumvent issues with partitioning during subsequent binding studies.

Described herein are substrates coated with one or more polymers capable of being attached to one or more different biomolecules and methods of making and using thereof. The methods for using the coated substrates provide numerous advantages over the art. For example, the substrate does not need to be activated, which saves the user time, cost, and complexity. Additionally, the methods for producing the coated substrates permit high-volume manufacturing of the substrates. In general, the coated substrates are stable and can be stored for extended (˜6 months) periods of time with little or no loss in binding capacity. Moreover, the coated substrates are slow to hydrolyze under acidic conditions, which permits the binding of various biomolecules under conditions that have not been described using prior art techniques for polymers such as, for example anhydride polymers.

### SUMMARY

Described herein are polymer-coated substrates for binding biomolecules and methods of making and using thereof. The advantages of the materials, methods, and articles described herein will be set forth in part in the description which follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. It will be appreciated that these drawings depict only typical embodiments of the materials, articles, and methods described herein and are therefore not to be considered limiting of their scope.

FIG. 1 shows a schematic representation of the two step modification procedure used to derivatize surfaces with maleic anhydride copolymers.

FIG. 2 shows a plot of the fluorescence signal (from a Cy3-streptavidin, biotin-amine assay) as a function of hydrolysis time for two different maleic anhydride copolymers.

FIG. 3 shows the results of a storage stability experiment on slides coated with poly(ethylene-alt-maleic anhydride) (EMA). The data indicate that EMA is stable for at least 4 months when stored dessicated at room temperature.

FIG. 4A shows the results of a Corning LID (a microplate-based, waveguide resonant grating detection platform) assay (binding of streptavidin to immobilized biotin-amine groups) performed on an EMA coated LID microplate.

FIG. 4B shows the results of an SPR experiment comparing the specificity of binding on MAMVE and SMA coated gold chips.

FIG. 5 shows the results of vancomycin binding experiments performed on Biacore CM5 and EMA coated gold chips using SPR detection.

FIG. 6 shows a competitive inhibition binding experiment performed on EMA coated gold chips using SPR detection.

FIG. 7 shows the results of a competitive inhibition binding experiment performed on EMA coated microplates using Corning LID detection.

FIG. 8 shows the relative amount of protein immobilized on EMA as a function of immobilization pH for 6 different proteins as determined using Corning LID detection on EMA coated microplates.

FIG. 9 shows the relative amount of protein immobilized on EMA coated microplates as a function of protein concentration.

FIG. 10 shows the results of an antibody-antibody binding assay performed on an EMA coated LID microplate.

FIG. 11 shows the Corning LID detection of the binding of fluorescein-biotin to EMA coated LID microplates presenting streptavidin.

FIG. 12 shows the Corning LID experiment of the binding of biotin to streptavidin immobilized on EMA.

FIG. 13A shows the Corning LID experiment of the binding of the drug digitoxin to human serum albumin immobilized on EMA.

FIG. 13B shows the results of a digitoxin titration series.

FIG. 14A shows the Corning LID experiments of the binding of the drug warfarin to human serum albumin immobilized on EMA.

FIG. 14B shows the results of a negative control experiment in which warfarin was replaced with a buffer blank.

FIG. 15 shows the results of drag binding experiments to human serum albumin immobilized on EMA using surface plasmon resonance detection.

FIG. 16 shows an SPR experiment examining the non-specific binding of proteins to maleic anhydride copolymer modified gold surfaces blocked with ethanolamine (EA) and various dextrans. Only the surface blocked with DEAE-dextran shows significantly increased resistance to the binding of proteins.

FIG. 17 shows an SPR experiment comparing the binding of anti-IgG to surfaces with immobilized IgG that were blocked with either ethanolamine or DEAE dextran. This experiment shows that DEAF dextran does not interfere with anti-IgG binding.

### DETAILED DESCRIPTION

Before the present materials, articles, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

By “contacting” is meant an instance of exposure by close physical contact of at least one substance to another substance.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and biomolecules are disclosed and discussed, each and every combination and permutation of the polymer and biomolecule are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### I. Coated Substrates

Described herein are polymer-coated substrates for binding biomolecules. In one aspect, described herein is a substrate comprising a first tie layer and a first polymer, wherein the first polymer comprises one or more functional groups that can bind a biomolecule to the substrate, wherein the tie layer is attached to the substrate, wherein the tie layer attaches the first polymer to the substrate.

In one aspect, the tie layer is attached to the outer surface of the substrate. The term “outer surface” with respect to the substrate is the region of the substrate that is exposed and can be subjected to manipulation. For example, any surface on the substrate that can come into contact with a solvent or reagent upon contact is considered the outer surface of the substrate. The substrates that can be used herein include, but are not limited to, a microplate or a slide. In one aspect, when the substrate is a microplate, the number of wells and well volume will vary depending upon the scale and scope of the analysis.

In one aspect, the substrate comprises a plastic, a polymeric or co-polymeric substance, a ceramic, a glass, a metal, a crystalline material, a noble or semi-noble metal, a metallic or non-metallic oxide, a transition metal, or any combination thereof. Additionally, the substrate can be configured so that it can be placed in any detection device. In one aspect, sensors can be integrated into the bottom/underside of the substrate and used for subsequent detection. These sensors could include, but are not limited to, optical gratings, prisms, electrodes, and quartz crystal microbalances. Detection methods could include fluorescence, phosphorescence, chemiluminescence, refractive index, mass, electrochemical. In one aspect, the substrate is a Corning LID microplate.

The substrates described herein have a tie layer attached to the substrate. The term “attached” as used herein is any chemical interaction between two components or compounds. The type of chemical interaction that can be formed when the first tie layer compound is attached to the substrate will vary depending upon the material of the substrate and the compound used to produce the first tie layer. In one aspect, the first tie layer can be covalently and/or electrostatically attached to the substrate. In one aspect, when the first tie layer is electrostatically attached to the substrate, the compound used to make the first tie layer is positively charged and the outer surface of the substrate is treated such that a net negative charge exists so that first tie layer compound and the outer surface of the substrate form an electrostatic bond. In another aspect, the first tie layer compound can form a covalent bond with the outer surface of the substrate. For example, the outer surface of the substrate can be derivatized so that there are groups capable of forming a covalent bond with the first tie layer compound.

In one aspect, the first tie layer is derived from a compound comprising one or more reactive functional groups. The phrase “derived from” with respect to the first tie layer is defined herein as the resulting residue or fragment of the first tie layer compound when it is attached to the substrate. The functional groups permit the attachment of the first polymer to the first tie layer. In one aspect, the functional groups of the first tie layer compound comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof In one aspect, the first tie layer is derived from a straight or branched-chain aminosilane, aminoalkoxysilane, aminoalkylsilane, aminoarylsilane, aminoaryloxysilane, or a derivative or salt thereof. In a further aspect, the first tie layer is derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoane. In another aspect, the first tie layer is derived from a polyamine such as, for example, poly-lysine or polyethyleneimine.

In another aspect, the first tie layer comprises a self-assembled monolayer (SAM). In one aspect, when the substrate surface is composed of gold, the SAM comprises an amine-terminated alkanethiol. In this aspect, the self-assembled monolayer comprises 11-mercaptoundecylamine.

A first polymer comprising one or more functional groups that can bind a biomolecule to the substrate is attached to the first tie layer. The “functional group” on the first polymer or any polymer described herein permits the attachment of the first polymer to the first tie layer or the biomolecule. Similarly, the functional groups present on the first or second tie layer permit the attachment of the first polymer or second polymer to the first or second tie layer, respectively. The first polymer or subsequent polymers can have one or more different functional groups. It is also contemplated that some first polymer may also be attached to the outer surface of the substrate as well as attached to the first tie layer. Alternatively, the first polymer may be in contact with the outer surface of the substrate and still be attached to the first tie layer. In one aspect, the first polymer can be covalently and/or electrostatically attached to the first tie layer. It is also contemplated that two or more different first polymers can be attached to the first tie layer.

The first polymer can be water-soluble or water-insoluble depending upon the technique used to attach the first polymer to the first tie layer. The first polymer can be either linear or non-linear. For example, when the first polymer is non-linear, the first polymer is a dendritic polymer. The first polymer can be a homopolymer or a copolymer.

In one aspect, the first polymer comprises at least one electrophilic group susceptible to nucleophilic attack. Not wishing to be bound by theory, when the first tie layer possesses a nucleophilic group that reacts with the electrophilic group of the polymer to form a covalent bond, a negative charge is produced at the first polymer. The negative charge at the first polymer layer can then facilitate the formation of an electrostatic bond between the first polymer and a biomolecule, a second tie layer, or a second polymer, all of which will be discussed in detail below. Alternatively, one or more electrophilic groups present on the first polymer layer can form a covalent bond with a biomolecule, a second tie layer compound, or a second polymer. In the case when a biomolecule is attached to the first polymer, the presence of specific side chains in the polymer (e.g. ethylene glycol) can help prevent non-specific binding of the biomolecule to the first polymer.

In one aspect, the first polymer comprises at least one amine-reactive group. The term “amine-reactive group” is any group that is capable of reacting with an amine group to form a new covalent bond. The amine can be a primary, secondary, or tertiary amine. In one aspect, the amine-reactive group comprises an ester group, an epoxide group, or an aldehyde group. In another aspect, the amine-reactive group is an anhydride group.

In one aspect, the first polymer comprises a copolymer derived from maleic anhydride and a first monomer. In this aspect, the amount of maleic anhydride in the first polymer is from 5% to 50%, 5% to 45%, 5% to 40%, 5% to 35%, 5% to 30%, 5% to 25%, 10% to 50%, 15% to 50%, 20% to 50%, 25% to 50%, or 30% to 50% by stoichiometry (i.e., molar amount) of the first monomer. In one aspect, the first monomer selected improves the stability of the maleic anhydride group in the first polymer. In another aspect, the first monomer reduces nonspecific binding of the biomolecule to the substrate. In a further aspect, the amount of maleic anhydride in the first polymer is about 50% by stoichiometry of the first monomer. In another aspect, the first monomer comprises styrene, tetradecene, octadecene, methyl vinyl ether, triethylene glycol methyl vinyl ether, butylvinyl ether, divinylbenzene, ethylene, acrylamide, dimethylacrylamide, pyrolidone, a polymerizable oligo(ethylene glycol) or oligo(ethylene oxide), or a combination thereof.

In one aspect, the first polymer comprises, poly(vinyl acetate-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), or a combination thereof. In another aspect, the first polymer is polyethylene-alt-maleic anhydride).

The amount of first polymer attached to the first tie layer can vary depending upon the selection of the first tie layer, the first polymer, and the intended use of the substrate. In one aspect, the first polymer comprises at least one monolayer. In another aspect, the first polymer has a thickness of about 10 Å to about 2,000 Å. In another aspect, the thickness of the first polymer has a lower endpoint of 10 Å, 20 Å 40 Å, 60 Å, 80 Å, 100 Å, 150 Å, 200 Å, 300 Å, 400 Å, or 500 Å and an upper endpoint of 750 Å, 1,000 Å, 1,250 Å, 1,500 Å, 1,750 Å, or 2,000 Å, where any lower endpoint can be combined with any upper endpoint to form the thickness range.

In one aspect, the first tie layer is aminopropylsilsesquioxane and the first polymer is poly(ethylene-alt-maleic anhydride).

In another aspect, the substrate further comprises a second tie layer and second polymer, wherein the second tie layer is attached to the first polymer, and the second polymer is attached to the second tie layer. Any of the first tie compounds described above can be used as the second tie compound. The nature of the attachment of the second tie layer to the first polymer and the second polymer to the second tie layer will vary depending upon the selection of materials. The second tie layer can be covalently or electrostatically attached to the first polymer. Alternatively, the second polymer can be covalently or electrostatically attached to the second tie layer. In one aspect, the second tie layer is covalently attached to the first polymer, and the second polymer is covalently attached to the second tie layer. It is contemplated that multiple tie layers and polymer layers can be applied to the first polymer once it is attached to the first tie layer.

The first and second tie layers can be prepared from the same or different compounds. Similarly, the first and second polymers can be the same or different as well. It is also contemplated that multiple tie layers and polymer layers can be attached to the first polymer depending upon the intended use of the substrate.

In one aspect, the second tie layer is derived from a polyamine or polyol. For example, the second tie layer can be ethylene diamine, ethylene glycol, or an oligoethylene glycol diamine. In another aspect, the second tie layer is derived from a diamine, a triamine, or a tetraamine.

Any of the first polymers described above can be used as the second polymer. In one aspect, the second polymer comprises at least one amine-reactive group such as, for example, an ester group, an epoxide group, an aldehyde group, or an anhydride group. In another aspect, the second polymer comprises polymaleic anhydride or a copolymer derived from maleic anhydride.

Prior to or subsequent to attaching the first polymer (or subsequent polymer layer), a linker can be optionally attached to the first polymer (or subsequent polymer layer). The term “linker” is any compound that can be attached to the polymer layer and possesses at least one group capable of coordinating with or binding to another molecule such as, for example, a biomolecule. The mechanism of coordination can be, for example, through a Lewis acid/base interaction, a Bronsted acid/base interaction, an ionic bond, a covalent bond, or an electrostatic interaction. In one aspect, the linker can possess a ligand that coordinates with an affinity tag (e.g. a hexahistidine tag) present in the biomolecule. For example, the linker can be a ligand that binds, chelates, or coordinates with a metal ion (e.g. Cu, Co, Ni) for the capture of histidine tagged proteins. In one aspect, the linker comprises N-(5-amino-1-carboxypentyl)iminodiacetic acid. Alternatively, the linker can possess a group that forms a hydrogen bond with the biomolecule. In another aspect, the linker can be an antibody that recognizes an antigen. In another aspect, the linker can be streptavidin for capture of biotinylated compounds. In yet another aspect, the linker can contain a thiol or disulfide group for capture of biomolecules via disulfide exchange reactions. Alternatively, the linker can contain groups reactive toward thiols (e.g. maleimide groups) for the binding of proteins through thiol groups such as cysteine. In another aspect, the linker can possess groups that promote the adhesion/binding of cells, such as the peptide sequence RGD. The linker can be attached to the polymer layer through any chemical interaction such as, for example, a covalent bond or an electrostatic interaction.

It is contemplated that one or more different biomolecules can be attached to the substrate to produce a variety of biological sensors. In one aspect, the biomolecule can be attached covalently or electrostatically to the first polymer (or subsequent polymer layer). The biomolecules may exhibit specific affinity for another molecule through covalent or non-covalent bonding. Examples of biomolecules useful herein include, but are not limited to, a natural or synthetic oligonucleotide, a natural or modified/blocked nucleotide/nucleoside, a nucleic acid (DNA) or (RNA), a peptide comprising natural or modified/blocked amino acid, an antibody, a hapten, a biological ligand, a membrane protein, a lipid membrane, a small pharmaceutical molecule such as, for example, a drug, or a cell.

In one aspect, the biomolecule can be a protein. For example, the protein can include peptides, fragments of proteins or peptides, membrane-bound proteins, or nuclear proteins. The protein can be of any length, and can include one or more amino acids or variants thereof. The protein(s) can be fragmented, such as by protease digestion, prior to analysis. A protein sample to be analyzed can also be subjected to fractionation or separation to reduce the complexity of the samples. Fragmentation and fractionation can also be used together in the same assay. Such fragmentation and fractionation can simplify and extend the analysis of the proteins.

In one aspect, following attachment of the biomolecule to the polymer layer and prior to a ligand binding assay, the blocking of residual charged groups on the surface of the polymer can be performed to minimize nonspecific binding interactions between the surface and the ligand due to electrostatic interactions. The term “ligand” as used herein as any free biomolecule (e.g., protein, peptide, DNA, RNA, virus, bacterium, cell) or chemical compound (e.g., drug, small molecule, etc) that interacts or binds with an immobilized biomolecule or compound. Inadequate blocking can lead to high levels of non-specific binding of the ligand, making analysis of the results difficult. In one aspect, the blocking agent is attached to the polymer layer by contacting the surface of the polymer layer with a charged polymer or compound that has good non-specific binding properties itself. The charged compound negates a substrate surface of an opposite charge. In other words, it cancels or masks the influence of the substrate. In one aspect, a compound having a positive charge such as, for example, dextran (e.g. DEAE dextran), can reduce non-specific binding of proteins to a negatively charged, anhydride-modified surface.

### II. Methods for Preparing Coated-Substrates

Described herein are methods for producing a substrate comprising (1) attaching a first tie layer compound to the substrate and (2) attaching a first polymer to the first tie compound. The methods contemplate the sequential attachment of the first tie layer to the substrate followed by attaching the first polymer to the first tie layer. Alternatively, it is contemplated to attach the first polymer to the first tie layer followed by attaching the first tie layer/first polymer to the substrate.

The first tie layer and first polymer can be attached to the substrate using techniques known in the art. For example, the substrate can be dipped in a solution of the first tie compound or the first polymer. In another aspect, the first tie compound or first polymer can be sprayed, vapor deposited, screen printed, or robotically pin printed or stamped on the substrate. This could be done either on a fully assembled substrate or on a bottom insert (e.g., prior to attachment of the bottom insert to a holey plate to form a microplate). The thickness of the first polymer layer (and subsequent polymer layers) can vary depending upon the intended use of the substrate. Thus, different techniques can be employed to vary the thickness of the polymer layer.

Using similar techniques, after the first polymer is attached to the first tie layer, a second tie layer compound can be attached to the first polymer, followed by attaching a second polymer to the second tie layer compound. Similar to above, the second tie layer and second polymer can be attached sequentially or concurrently to the first polymer using techniques known in the art. In other aspects, a linker or blocking agent can be attached the first polymer (or subsequent polymer layers) using the techniques outlined above.

Once the first polymer or subsequent polymer layers have been attached to the substrate, one or more biomolecules can be attached to the polymer layer using the techniques presented above. The reaction kinetics of attaching the biomolecule to the polymer layer is generally fast. In one aspect, the biomolecule is attached to the substrate in a sufficient amount under about 1 hour, 30 minutes or 15 minutes.

The amount of biomolecule that can be attached to the polymer layer can vary depending upon, for example, the size and the isoelectric point of the biomolecule. Due to the hydrolytic stability of the coated substrate, the biomolecule can be attached to the polymer layer under a variety of conditions that would otherwise not been possible. For example, the coated substrates described herein can bind many proteins in acidic conditions. In one aspect, the biomolecule is attached to the first polymer at a pH of from about 0.5 to 1 pH units below the isoelectric point of the biomolecule.

### III. Methods of Use

Described herein are methods for performing an assay of a bioactive agent, comprising (1) contacting the ligand with a substrate comprising a first tie layer, a first polymer, and a biomolecule, wherein the tie layer attaches the first polymer to the substrate, and wherein the biomolecule is attached to the first polymer, wherein the ligand is bound to the biomolecule after the contacting step, and (2) detecting the bound ligand.

Any of the substrates described herein with one or more biomolecules attached thereto can be used to bind a ligand and ultimately detect the bound ligand. The binding of the ligand to the substrate involves a chemical interaction between the biomolecule and the ligand; however, it is possible that an interaction may occur to some extent between the polymer layer and the ligand. The nature of the interaction between the biomolecule and the ligand will vary depending upon the biomolecule and the ligand selected. In one aspect, the interaction between the biomolecule and the ligand can result in the formation of an electrostatic bond, a hydrogen bond, a hydrophobic bond, or a covalent bond. In another aspect, an electrostatic interaction can occur between the biomolecule and the ligand.

The ligand can be any naturally-occurring or synthetic compound. Examples of ligands that can be bound to the biomolecules on the substrate include, but are not limited to, a drug, an oligonucleotide, a nucleic acid, a protein, a peptide, an antibody, an antigen, a hapten, or a small molecule (e.g., a pharmaceutical drug). Any of the biomolecules described above can be a ligand for the methods described herein. In one aspect, a solution of one or more ligands is prepared and added to one or more wells that have a biomolecule attached to the outer surface of the microplate. In this aspect, it is contemplated that different biomolecules can be attached to different wells of the microplate; thus, it is possible to detect a number of different interactions between the different biomolecules and the ligand. In one aspect, a protein can be immobilized on the microplate to investigate the interaction between the protein and a second protein or small molecule. Alternatively, a small molecule can be immobilized on the microplate using the techniques described herein to investigate the interaction between the small molecule and a second small molecule or protein. In one aspect, when the substrate is a microplate, the assay can be a high-throughput assay.

Once the ligand has been bound to the biomolecules on the substrate, the bound ligand is detected. One of the advantages of the substrates described herein is that non-specific binding of the ligand is reduced.

In one aspect, the bound ligand is labeled for detection purposes. Depending upon the detection technique used, in one aspect, the ligand can be labeled with a detectable tracer prior to detection. The interaction between the ligand and the detectable tracer can include any chemical or physical interaction including, but not limited to, a covalent bond, an ionic interaction, or a Lewis acid-Lewis base interaction. A “detectable tracer” as referred to herein is defined as any compound that (1) has at least one group that can interact with the ligand as described above and (2) has at least one group that is capable of detection using techniques known in the art. In one aspect, the ligand can be labeled prior to immobilization. In another aspect, the ligand can be labeled after it has been immobilized. Examples of detectable tracers include, but are not limited to, fluorescent and enzymatic tracers.

In another aspect, detection of the bound ligand can be accomplished with other techniques including, but not limited to, fluorescence, phosphorescence, chemilumenescence, bioluminescence, Raman spectroscopy, optical scatter analysis, mass spectrometry, etc. and other techniques generally known to those skilled in the art.

In one aspect, the immobilized ligand is detected by label-independent detection or LID. Examples of LID include, but are not limited to, surface plasmon resonance or a resonant waveguide gratings (e.g. Corning LID system). As practiced in the prior art, substrates for LID assays have limitations. Assays using label-free detection platforms are typically performed using a two step procedure: (i) immobilization of one of the binding partners (typically a protein) on the surface of the sensor; and (ii) binding of a ligand (e.g., drug, protein, oligonucleotide, etc) to the immobilized protein. Traditionally, the coupling of biomolecules to surfaces involves the activation of carboxylic acid groups on the surface to reactive N-hydroxysuccinimide (NHS) esters, which are then coupled to amino groups on the protein of interest. This method has been successfully used and commercialized by Biacore, Affinity Biosensors, and Artificial Sensing Instruments for their respective LID platforms. While effective, the activation step is time consuming and involves the handling and use of somewhat toxic chemicals. An alternative to this approach involves the use of “preactivated” chemistries. For example, surfaces presenting aldehyde groups have been used to bind biomolecules. However, a reduction step is required after coupling to stabilize the resulting Schiff base. Surfaces with epoxide and isocyanate functionalities have also been used, however, the epoxide group is relatively slow to react and therefore requires long incubation times under very basic conditions, while the isocyanate group is extremely reactive and therefore presents storage stability issues. The coated substrates described herein address the limitations of current LID platform technology.

### EXAMPLES

The following examples are put forth so as to provide those of ordinary skill in the art with a complete disclosure and description of how the materials, articles, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### A. Preparation of Coated Surfaces

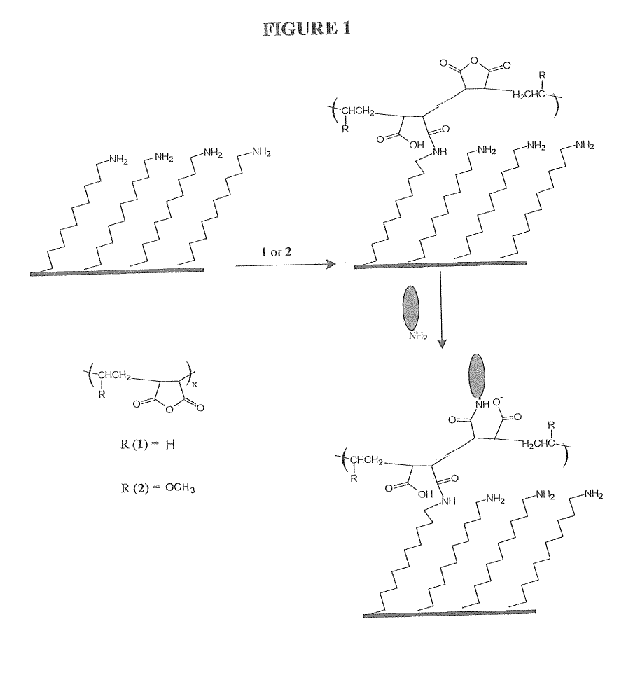

Surfaces (including glass, inorganic oxides, and gold) were modified with maleic anhydride copolymers using a two step modification procedure depicted in FIG. 1 and described in detail below.

### EMA on Inorganic Oxides

### Materials

Bare glass slides or Corning GAPS slides
Aminopropylsilsesquioxane (“APS”) (Gelest catalog #WSA-9911)
Poly(ethylene-alt-maleic anhydride) (“EMA”) (Aldrich catalog # 18,805-0)
N-methylpyrolidone (NMP)
Isopropanol (IPA) (low water content)
Absolute Ethanol
50 mL glass coplin staining jar

### Procedure

(If Corning GAPS slides are used, skip steps 1-2.)
1. Clean the bare glass slides by treating in an O₂ plasma chamber for 5 min (1 T, 250 Watts); alternatively, treat the slides in a UV ozone chamber for 3 minutes.
2. In a 50 mL coplin staining jar, react the slides for 10 min with a 5% (vol/vol) solution of aminopropylsilsesquioxane in water; rinse the slides with water, then ethanol, and dry with nitrogen.
3. React the slides for 10 min with 1 mg/mL EMA in 10% NMP: 90% IPA in a 50 mL glass coplin staining jar. Dissolve the EMA in 100% NMP at 10 mg/mL and then dilute 10× with IPA; (when doing the dilution, it is important to add the NMP to the IPA otherwise a precipitate may form.) After 10 min, rinse the surface with ethanol and dry with nitrogen.
4. Store the slides in a dessicator at room temperature until ready to use.

### EMA on Gold

### Materials

Biacore bare Au sensor chip
11-mercatptoundecylamine (Dojindo)
Poly(ethylene-alt-maleic anhydride) (“EMA”) (Aldrich catalog # 18,805-0)
N-methylpyrolidone (NMP)
Isopropanol (IPA) (low water content)
Dimethylsulfoxide (DMSO)
Absolute Ethanol

### Procedure

1. Clean the bare gold chip by rinsing with ethanol and water; dry under a stream of nitrogen.
2. Soak the chip for 1 hour in a 1 mM ethanolic solution of 11-mercaptoundecylamine; rinse the chip with ethanol and water, and dry under a stream of nitrogen.
3. React the slides for 10 min with 1 mg/mL solution of EMA in 10% NMP: 90% IPA or 100% DMSO. After 10 min, rinse the chip with ethanol and dry with nitrogen.
   Synthesis of poly(triethyleneglycol methylvinyl ether-co-maleic anhydride) (“PEG-MA”)

Poly(triethyleneglycol methylvinyl ether-co-maleic anhydride) (“PEG-MA”) was synthesized by free radical polymerization of maleic anhydride and triethyleneglycol methylvinyl ether. A 50 mL round bottom flask was charged with 1.018 mL triethyleneglycol methylvinyl ether, 520 mg maleic anhydride, 3 mg AIBN, and 7 mL toluene. The mixture was allowed to react at 63° C. overnight. The polymer was isolated by precipitation from ether. FTIR analysis confirmed that the reaction was successful. Amine-presenting surfaces (e.g. gold chips derivatized with 11-mercaptoundecylamine or glass/silicon modified with aminopropylsilsesquioxane) were modified with PEG-MA by soaking in a 10 mg/mL solution of the polymer in methyl ethyl ketone with 0.1% (vol/vol) triethylamine for 30-60 minutes. The chips were rinsed with methylethyl ketone and ethanol, and dried under a stream of nitrogen.

### B. Characterization of Maleic Anhydride Copolymer Surfaces

Ellipsometry on Gold. Ellipsometry was used to characterize the attachment of poly(maleic anhydride-alt-methylvinyl ether) (MAMVE) to amine-presenting self-assembled monolayers (SAMs) on gold, and the subsequent attachment of amine-containing molecules to the reactive surface. The increase in thicknesses of SAMs presenting different functional groups after being reacted with MAMVE are tabulated below (Table 1). Among the surfaces tested, only SAMs presenting amine groups showed an increase in thickness. If the polymer is immobilized with the polymer backbone parallel to the surface, the expected increase in thickness is ˜6-7 Å, which corresponds to the observed increase in thickness. It was hypothesized that a monolayer of the polymer is conjugated to the SAM to form a comb-like structure.

TABLE 1
Ellipsometric increases in thickness (Δd) of different
SAMS after reaction with MAMVE and after subsequent
reaction with undecylamine (UA).

| SAM | Δd MAMVE (Å) | Δd UA (Å) |
|---|---|---|
| HSC₁₁NH₂ | 7.1 ± 1.1^{a} | 5.2 ± 0.8^{b} |
| HSC₁₆ | 0 | — |
| HSC₁₀COOH | 0 | — |
| HSC₁₁OH | 0 | — |

^{a}average of 8 samples
^{b}average of 3 samples

To investigate the amount of coupling to the anhydride-modified surface, the substrate was immersed in a solution of undecylamine (“UA”, 10 mM) in DMSO for 1.5 hours. After derivatization with UA, the thickness of the surface increased by ˜5 Å (Table 1). A packed monolayer of undecylamine would give an ellipsometric thickness of ˜17 Å; thus, the observed increase in thickness corresponds to ˜30% coverage of the surface.

In order to determine whether the attachment of MAMVE to the amine-SAM was covalent or electrostatic, experiments were performed to examine whether the observed increase in thickness was reversible or not. An irreversible increase in thickness would suggest covalent attachment; conversely, a reversible increase in thickness would suggest non-covalent attachment. It was found that there was no decrease in the thickness of the substrate after washing with acidic buffer (pH 3). In another experiment, MAMVE was hydrolyzed by stirring overnight in a solution of ammonia. The adsorption of this hydrolyzed polymer to the amine-presenting SAM resulted in an increase in thickness corresponding to ˜8.6 Å. This adsorption is probably due to electrostatic interactions between the negatively charged polymer and the positively charged surface. There was, however, no subsequent increase in thickness after reaction with undecylamine. Moreover, soaking the surface in an acidic buffer (pH 3) resulted in a large decrease in the thickness. At this pH, the carboxylate groups of the hydrolyzed polymer are protonated to form carboxyl groups, which would greatly decrease the affinity of the polymer for the surface and lead to desorption.

Ellipsometry on Inorganic Oxides. Ellipsometry was used to characterize the attachment of poly(ethylene-alt-maleic anhydride) (EMA) to silicon wafers coated with different inorganic oxides. Prior to attachment of EMA, the substrates were modified with a tie layer of aminopropylsilsesquioxane (APS) as described in section A above. Table 2 summarizes the results of these experiments and shows that significantly more EMA was deposited on SiO₂ relative to the other substrates. Analysis of the data indicates that the thickness of the EMA layer varied with the thickness (amount) of the APS tie layer, and SiO₂ had the thickest APS layer. Control experiments performed on SiO₂ surfaces with no APS layer showed no increase in ellipsometric thickness, indicating that EMA does not adhere to the surface in the absence of the adhesion layer. Note that in these experiments, only one set of conditions (optimized for SiO₂) was employed for coating of all substrates; no attempt was made to increase the APS thickness on other substrates. The nature of the solvent used for deposition of EMA had an influence on the thickness of the EMA layer. Specifically, use of a mixed NMP/IPA solvent resulted in a layer of EMA twice as thick as layers deposited from DMSO (see Table 3). Samples prepared using these two solvent systems were subjected to an aggressive (16 hour soak in DMSO) wash procedure. As shown in Table 4, essentially no change in ellipsometric thickness was observed for samples prepared from DMSO suggesting that the EMA was covalently attached to the surface. A loss in thickness of ˜30% was observed for samples prepared using NMP/IPA. When the NMP/IPA solvent was used, some EMA did bind to substrates with no APS layer (see Table 3). However, this material was removed after the extended soak in DMSO.

TABLE 2
Ellipsometric increases in thickness (Δd) after reaction
of different substrates with APS followed by EMA.

| Substrate | Δd APS (Å) | Δd EMA (Å) |
|---|---|---|
| SiO₂ | 11.0 | 14.9 |
| SiO₂ | 0.0 | 0.7 |
| Ta₂O₅ | 6.8 | 9.5 |
| Nb₂O₅ | 5.8 | 6.8 |
| TiO₂ | 5.7 | 6.0 |

TABLE 3
Ellipsometric increases in film thickness (Δd) after reaction of
SiO₂ substrates with EMA in different solvents.

| Solvent | Δd EMA (Å) (+APS) | Δd EMA (Å) (no APS) |
|---|---|---|
| DMSO | 14.9 +/− 0.2 | 0.7 +/− 0.2 |
| NMP/IPA | 27.8 +/− 0.3 | 5.2 +/− 1.7 |

TABLE 4
Ellipsometric thickness (Δd) of EMA films deposited on
SiO₂ from different solvents after 16 hour soak in DMSO.

| Solvent | Δd EMA (Å) (+APS) | Δd EMA (Å) (no APS) |
|---|---|---|
| DMSO | 17.0 +/− 0.5 | — |
| NMP/IPA | 19.0 +/− 0.4 | 1.1 +/− 0.5 |

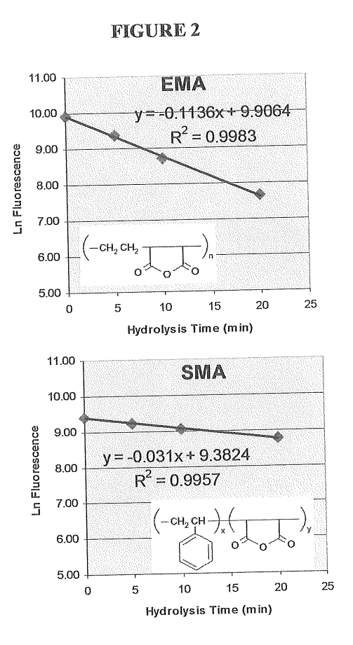

Hydrolytic Stability. A series of experiments was performed to evaluate the hydrolytic stability of thin films of different maleic anhydride copolymers. Corning GAPS slides were derivatized with one of the following polymers: EMA, poly(maleic anhydride-alt-1-octadecene) (“OMA”), poly(styrene-co-maleic anhydride) (“SMA”), MAMVE, or poly(triethyleneglycol methylvinyl ether-co-maleic anhydride) (“PEG-MA”). A 12-well gasket was affixed to the slide and a buffer solution (pH 7.4) was incubated with each well for varying amounts of time. A simple fluorescence binding assay was then performed in which biotin-peo-amine was first immobilized on the surface, followed by an incubation with a solution of Cy3-streptavidin. Any significant amount of hydrolysis of the reactive maleic anhydride groups would be manifest by a systematic decrease in fluorescence signal as a function of incubation time. Assuming a first order hydrolysis reaction, a plot of the ln(fluorescence signal) versus incubation time should yield a straight line with a slope equal to the negative reciprocal of the rate constant. FIG. 2 shows representative plots for thin films of EMA and SMA, and Table 5 summarizes the observed half lives of each maleic anhydride copolymer tested. The data show that the nature of the side chain influences the hydrolytic stability of the maleic anhydride. For example, SMA, which has a hydrophobic styrene side chain, has a significantly longer half life relative to EMA. The fact that the OMA copolymer, which has hydrophobic octadecene side chains, does not show the same hydrolytic stability as SMA, suggests that steric hindrance and/or the nature of the polymer repeat unit (alternating vs block) may also play a role in the hydrolytic stability of the copolymer.

TABLE 5
Observed half life of thin films of different maleic anhydride
copolymers as determined using a fluorescence binding assay.

| Thin Film | T_{1/2} (min)^{a} |
|---|---|
| SMA | 22-36 |
| PEGMA | 7-9 |
| EMA | 6.1 |
| OMA | 5.6 |
| MAMVE | 5.9 |

^{a}at pH 7.4

In a second series of experiments, grazing angle FTIR measurements on films of EMA were performed to directly and more quantitatively monitor the hydrolytic stability of EMA as a function of pH. Specifically, the decrease in the maleic anhydride carbonyl band as a function of time was used to determine the hydrolysis half life. Measurements were made on low-e glass microscope slides (Kevley Technologies) using a Nicolet Nexus 470 FTIR spectrometer equipped with a Harrick Seagull grazing angle accessory. Table 6 shows the results of these experiments. Note that at pH 5, the half life of EMA is significantly longer relative to pH 7.4 and 9.2. A comparison of the results in Tables 5 and 6 obtained on EMA at pH 7.4 show that the fluorescence data and the FTIR data are in relatively good agreement.

TABLE 6
Half life of thin films of EMA at different pHs as determined
using grazing angle FTIR.

| pH^{a} | T_{1/2} (min) |
|---|---|
| 5 | 289 |
| 7.4 | 4.7 |
| 9.2 | 0.8 |

^{a}Buffers were 15 mM acetate (pH 5), phosphate buffered saline (pH 7.4), 100 mM borate (pH 9.2)

Grazing angle FTIR was also used to monitor the hydrolytic stability of thin films of EMA as a function of relative humidity. These experiments showed that thin films of EMA have a half life of ˜6 hours at 100% relative humidity (23° C.); it is expected that the half life would be longer under more typical ambient conditions (˜65% relative humidity).

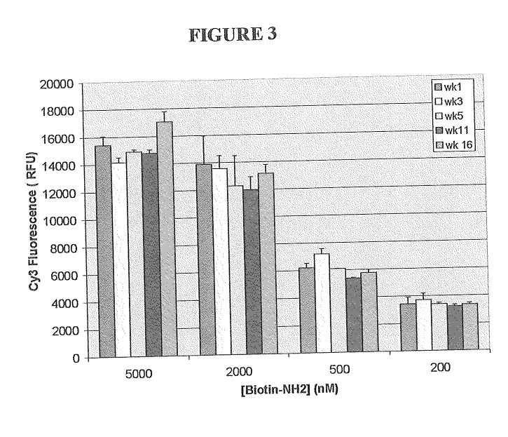

Storage Stability. The storage stability of thin films of EMA on GAPS slides was evaluated over a 4 month period of time. A set of EMA slides was prepared and stored dessicated at room temperature. The performance of a fluorescence assay as a function of storage time was evaluated. The assay consisted of the immobilization of biotin-peo-amine in a dilution series (200 nM-5 μM) followed by incubation with a fixed (100 nM) concentration of Cy3-streptavidin. Any significant amount of hydrolysis of the reactive maleic anhydride groups would be manifest by a systematic decrease in fluorescence signal as a function of storage time. Analysis of the results of these experiments (see FIG. 3) shows no significant decrease in performance for the 4 month period tested, indicating that the relatively simple storage conditions employed were effective.

### C. Binding Assays

A series of experiments was performed to demonstrate the utility of surfaces modified with maleic anhydride copolymers for label-free binding assays. These experiments utilized either Biacore surface plasmon resonance (SPR) or Corning LID detection.

### Small Molecule/Protein

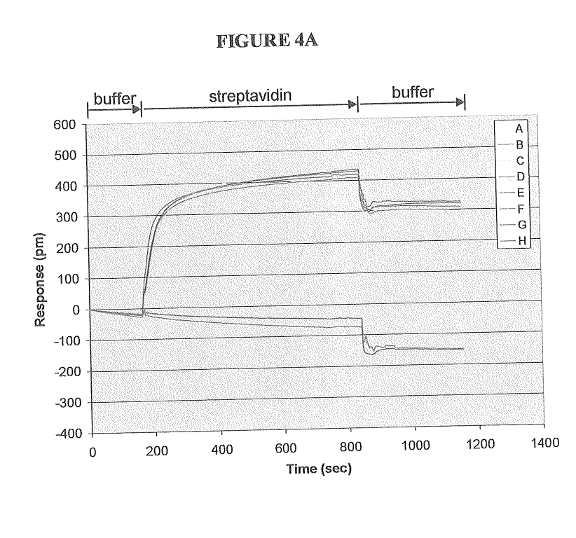

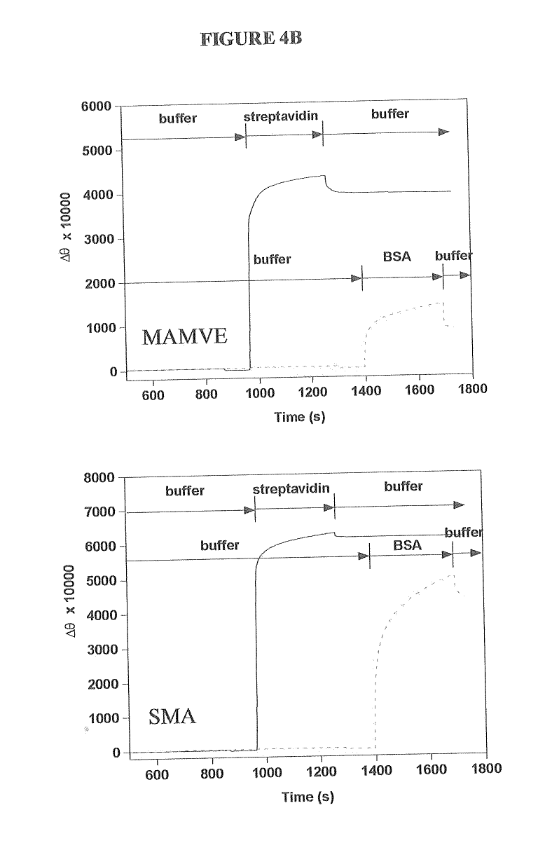

The performance of EMA in a small molecule/protein binding assay was tested using a biotin/streptavidin model system and Coming LID detection. Biotin was immobilized in rows A-G of a Corning LID microplate by incubating each well with 75 uL of a 10 uM solution of biotin-peo-amine in borate buffer (150 mM, pH 9) for 30 minutes; row H was reacted with ethanolamine (200 mM in borate buffer, pH 9) to serve as a negative control. The plate was docked in the LID instrument and the binding of streptavidin (100 nM in phosphate buffered saline (PBS)) was monitored as a function of time as shown in FIG. 4A. An average response of 465 pm was observed for six wells with a standard deviation of ˜3%. Analysis of the data indicates that the binding of streptavidin is specific because no binding was observed in the wells derivatized with ethanolamine (row H).

The binding of proteins to ligands immobilized on two different maleic anhydride copolymers, MAMVE and styrene-maleic anhydride (SMA), was also examined Biacore SPR detection was used for these experiments in which biotin was immobilized on the surfaces by injecting solutions of 5-(biotinamido)pentylamine over each surface. Solutions of streptavidin (1 μM) or BSA (as a control to test specificity) were then injected over the surfaces. FIG. 4B shows the amounts of binding of streptavidin and BSA to biotin groups immobilized on MAMVE and SMA. The data show that the binding of streptavidin to biotin-groups immobilized on MAMVE was specific. These data also show that the amount of non-specific binding of proteins on surfaces presenting styrene side chains was considerably greater than that on surfaces presenting methyl ethers. Non-specific binding of proteins to surfaces such as those presenting hydrophobic aromatic groups is well documented; the inertness of surfaces presenting —OCH₃ groups to non-specific adsorption has also been observed (13).

### Small Molecule/Small Molecule

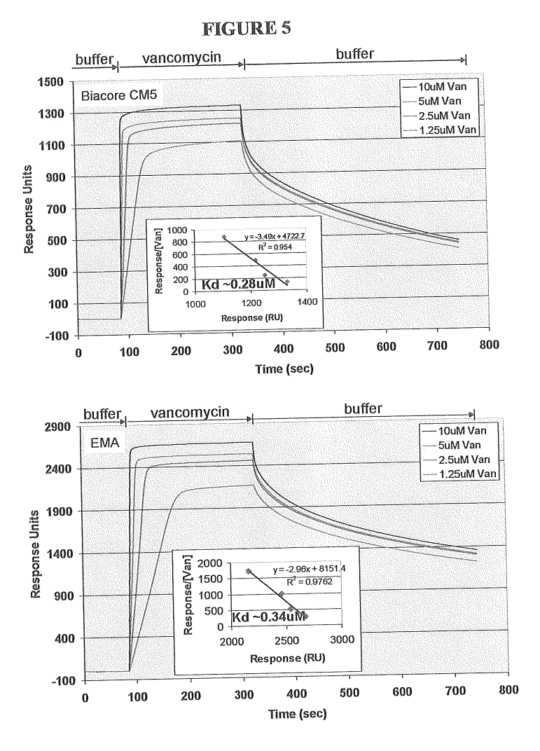

To demonstrate the utility of maleic anhydride copolymers for monitoring small molecule/small molecule interactions, the specific binding and competitive inhibition of the drug vancomycin (1486 Da) was evaluated on maleic anhydride modified surfaces presenting the peptide sequence Lys-D-Ala-D-Ala. (Vancomycin is an antibiotic that binds to the C-terminal D-Ala-D-Ala group of Gram-positive bacterial cell wall precursors and inhibits cell wall synthesis.) Experiments were performed on the Biacore 2000 SPR instrument using gold sensor chips modified with either EMA or poly(triethylene glycol-co-maleic anhydride) (“PEG-MA”); for comparison, experiments were also performed on Biacore's CM5 surface. FIG. 5 shows the sensorgrams for the binding of vancomycin to Lys-D-Ala-D-Ala immobilized on CM5 and EMA, respectively. As can be seen in the figure, the amount of binding was dose dependent; Scatchard analysis of the data gave observed Kd values of 0.28 μM and 0.34 μM, respectively (see Table 7). These results are similar to the Kd of ˜1 μM reported in the literature for these compounds measured in solution (12).

TABLE 7
Summary of vancomycin binding experiments performed
on three different surfaces using SPR detection.

| | Total Signal | | Nonspecific Binding |
|---|---|---|---|
| | (RU)^{a} | Kd (μM)^{b} | (RU)^{c} |
| CM5 | 1251 | 0.27 | 3 |
| EMA^{d} | 2538 | 0.34 | 290 |
| PEG- | 2006 | 1.3 | 128 |
| MA^{d} | | | |

^{a}Measured for 5 μM vancomycin
^{b}From a Scatchard analysis using vancomycin concentrations of 10 μM, 5 μM, 2.5 μM, 1.25 μM
^{c}Measured for 5 uM vancomycin + 500 μM DADA
^{d}Immobilization of Lys-D-Ala-D-Ala was performed outside of the Biacore

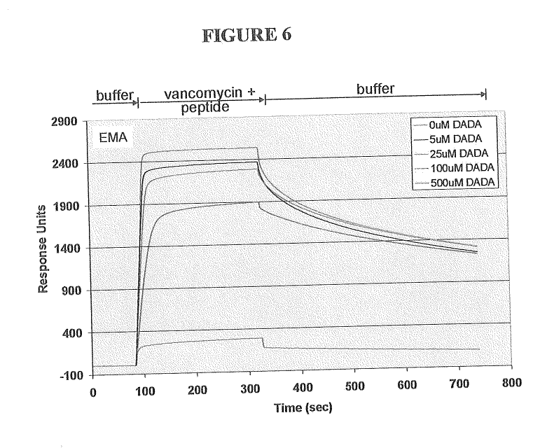

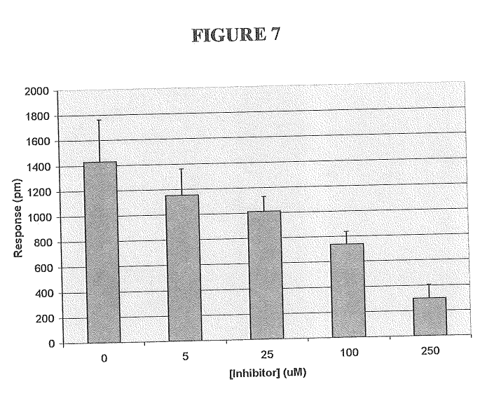

To test the specificity of the interaction, a competitive binding assay was performed in which the surface was incubated with a fixed (5 μM) concentration of vancomycin in the presence of varying concentrations of the peptide Lys-D-Ala-D-Ala. As shown in FIG. 6, the binding of vancomycin was inhibited in a dose dependent manner by the addition of the peptide, suggesting that the binding of vancomycin is specific. Both maleic anhydride copolymer surfaces tested showed binding specificity>90%. The amount of nonspecific binding in this assay was ˜2× lower on PEG-MA relative to EMA; this observation is consistent with the fact that oligo(ethylene glycol) groups have been shown to effectively inhibit nonspecific binding of proteins (13). Similar competitive inhibition experiments were performed on a Corning LID microplate coated with EMA (see FIG. 7). These studies showed that the vancomycin binding signal was reduced 83% in the presence of excess (250 uM) Lys-D-Ala-D-Ala.

### Protein/Protein

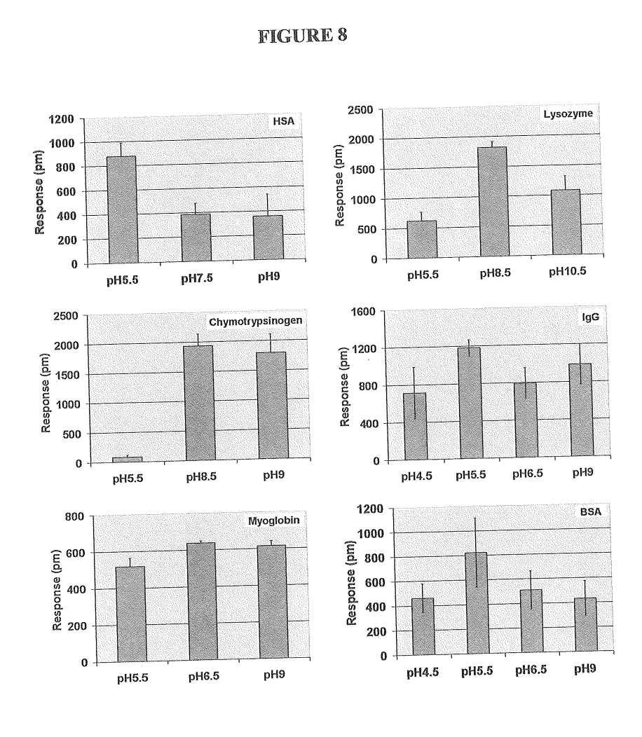

Experiments were performed on the Corning LID platform to demonstrate that proteins of varying size and isoelectric point (pI) can be immobilized on EMA. A total of 7 different proteins (lysozyme, chymotrypsinogen, human serum albumin (HSA), bovine serum albumin (BSA), myoglobin, streptavidin, and human IgG) were tested (see Table 8). FIG. 8 shows plots of the relative amount of protein bound (expressed in terms of pm shift in resonance signal) as a function of buffer pH. Although good levels of immobilization were achieved at several different pH values, in general, higher levels of immobilization were achieved using an immobilization buffer at a pH ˜0.5-1 unit below the pI of the protein. This observation is consistent with an electrostatic concentration effect in which the protein (positively charged below its pI) interacts strongly with the surface (negatively charged due to the presence of carboxylic acid groups generated from the hydrolysis of maleic anhydride), thereby enhancing the coupling efficiency.

TABLE 8
Properties of proteins immobilized on EMA

| Protein | Size (kDa) | pI |
|---|---|---|
| Lysozyme | 14 | 9.5 |
| Myoglobin | 17.6 | 7.2 |
| Chymotryp | 25 | 9 |
| Streptavidin | 60 | 5 |
| BSA | 66 | 5.9 |
| HSA | 66 | 5.5 |
| IgG | 150 | 6-8 |

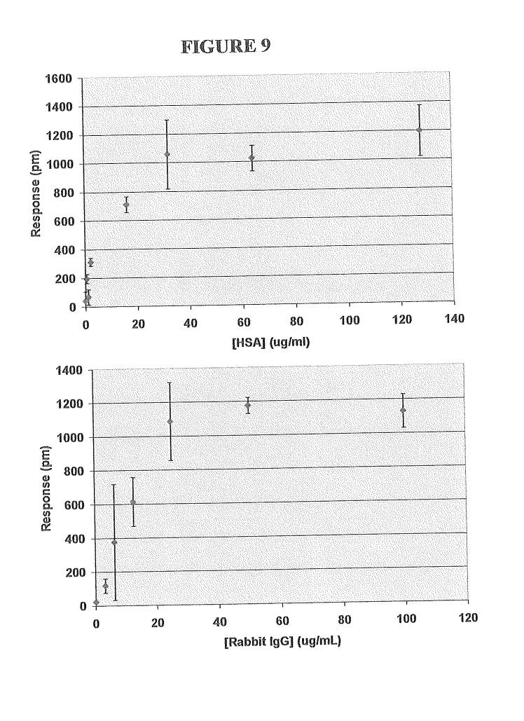

Corning LID experiments using microplates coated with EMA were also performed to investigate the influence of protein concentration on the amount of protein immobilized. The proteins chosen for this study were HSA and IgG. Concentrations of 0-128 μg/mL were tested in a buffer with a pH optimized for maximum binding. Binding was allowed to occur for 15 minutes, followed by a wash with buffer and a 5 minute incubation in 200 mM ethanolamine in borate buffer (150 mM, pH 9). This ethanolamine wash step is used to i) inactivate any residual reactive maleic anhydride groups; ii) remove nonspecifically bound protein from the surface. FIG. 9 shows representative data for the immobilization of HSA and IgG as a function of concentration. Saturation coverage was reached for concentrations≧˜30 ug/mL. Control wells that were blocked with ethanolamine prior to incubation with HSA or IgG showed low levels of binding, suggesting that the binding of the proteins to EMA is covalent, and demonstrating that the nonspecific binding of proteins to EMA was low

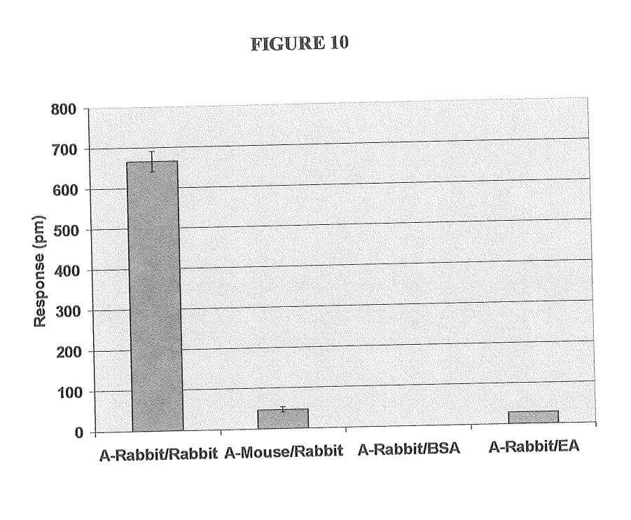

An antibody-antibody assay was chosen as a representative example of a protein-protein interaction. Specifically, a polyclonal rabbit IgG was immobilized in multiple wells of a Corning LID microplate; as negative controls, additional wells were derivatized with BSA or ethanolamine (EA). A solution of a polyclonal anti-rabbit (“a-rabbit”) antibody was incubated with each well and the amount of binding was quantified using the Corning LID platform. FIG. 10 shows a summary of the results. High signal was observed for the binding of the anti-rabbit antibody to the immobilized rabbit IgG; the amount of binding was reproducible with a CV of ˜4%. Incubation of wells presenting rabbit IgG with an anti-mouse antibody resulted in very low levels of binding; very little, if any, binding was observed when a solution of anti-rabbit antibody was incubated with wells presenting a generic protein (BSA) or wells blocked with ethanolamine. These data demonstrate that the a-rabbit/rabbit binding interaction is highly specific, and that the EMA surface shows minimal amounts of nonspecific binding.

### Protein/Small Molecule

The ability to detect the binding of small molecules to proteins is of great interest for drug discovery applications. Experiments were performed to demonstrate that i) proteins immobilized on surfaces modified with maleic anhydride copolymers retain their functionality and can be used for small molecule binding assays; ii) nonspecific binding of small molecules to EMA is low. Two model systems were chosen for these studies: the binding of fluorescein-biotin or biotin to streptavidin, and the binding of drugs to human serum albumin.

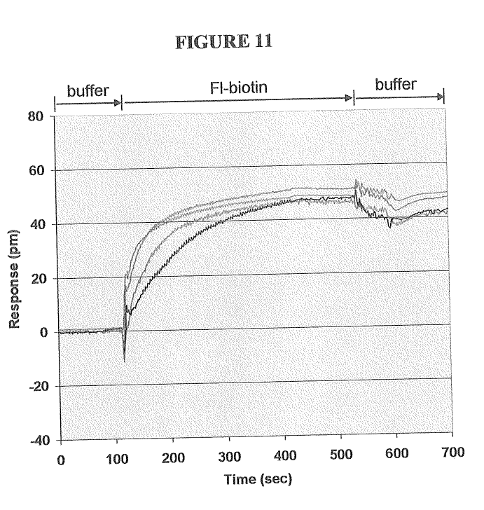

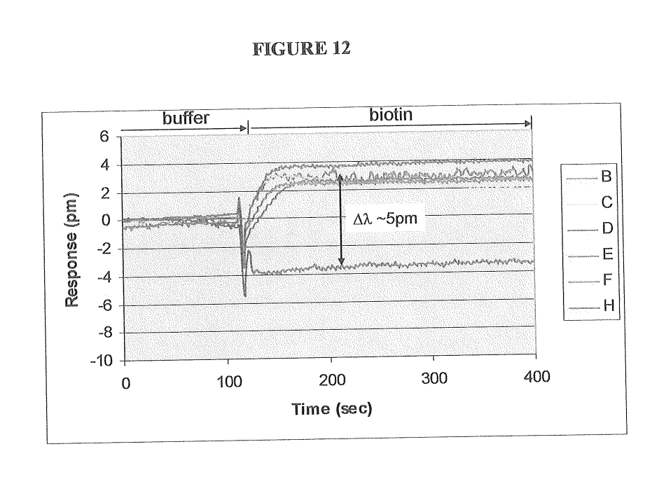

Streptavidin was immobilized on an EMA coated LID microplate by incubating the wells with a solution of 25 ug/mL of the protein in an acetate buffer (20 mM, pH 5.5) for 15 minutes. As negative controls, additional wells were blocked with ethanolamine. The binding of the small molecule fluorescein-biotin (“F1-biotin”, 831 Da) was monitored as a function of time in the LID instrument. A 100 nM solution of F1-biotin in PBS was introduced into each well and allowed to bind for several minutes. FIG. 11 shows a plot of the results. The data have been corrected for bulk index of refraction effects by subtracting out the response from the negative control well. A response of 48 pm +/−2 pm was observed for the binding of fluorescein-biotin, with a signal-to-noise ratio of >200. FIG. 12 shows the results of a similar experiment that was performed with the small molecule biotin. Relative to the negative control well (well H) that contained no streptavidin, the positive control wells (B-F) showed a response of ˜5 pm for the binding of biotin, with a signal-to-noise ratio of ˜50. This experiment was repeated in each column of the plate with similar results. These results demonstrate that small molecule binding experiments can be performed on proteins immobilized on EMA

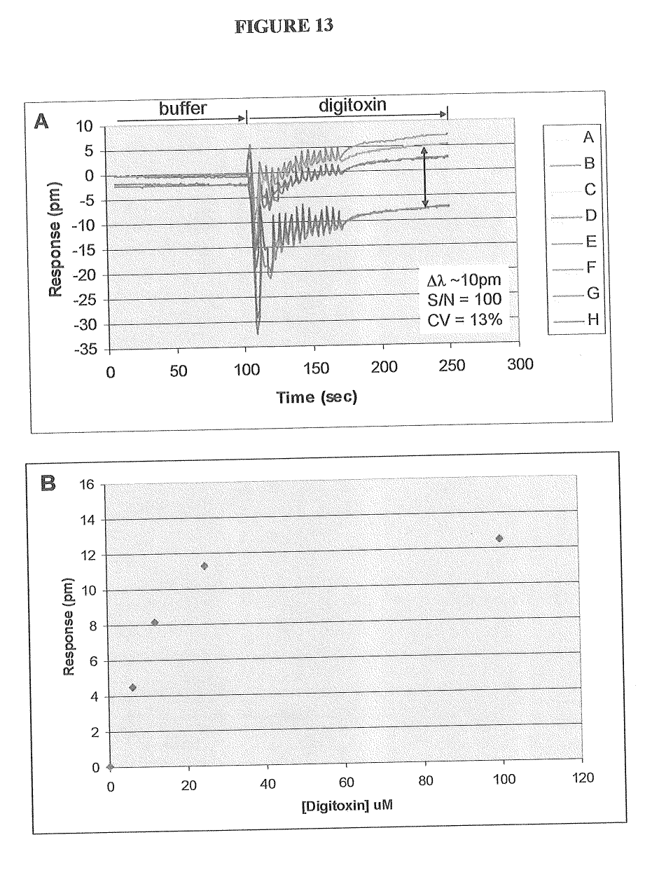

In another series of experiments, the binding of the drags digitoxin (765 Da) and warfarin (308 Da) to human serum albumin (HSA, a protein involved in the transport of drugs in the blood)) immobilized on 96-well microplates coated with EMA was measured using LID detection. HSA was immobilized on the sensor surface by incubating the wells with a solution of 60 ug/mL of the protein in an acetate buffer (20 mM, pH 5.5) for 15 minutes. As negative controls, additional wells were blocked with ethanolamine After thorough washing with PBS buffer and water, residual reactive groups were blocked by incubating the wells for 10 minutes with ethanolamine (200 mM, pH 9.2). The plate was docked in the LID instrument and equilibrated with 100 uL/well of a buffer solution (97% PBS/3% DMSO). 100 uL of digitoxin (200 uM) in an aqueous buffer solution (97% PBS/3% DMSO) was added to each well, mixed well, and allowed to bind for several minutes. As shown in FIG. 13A, relative to the negative control wells (G and H) which contained no immobilized HSA, the positive control wells (A-F) showed a reproducible shift in response of ˜10 pm. In a separate set of experiments, the binding of digitoxin was shown to be dose dependent and saturable (see FIG. 13B); concentrations as low as 6 uM (which would mean ˜30% of the binding sites would be occupied, based on the reported equilibrium dissociation constant (Kd) for the binding of digitoxin to HSA) were successfully detected. Scatchard analysis of the data indicated a Kd of 16 uM, which is in good agreement with the reported Kd of 16.5 uM.

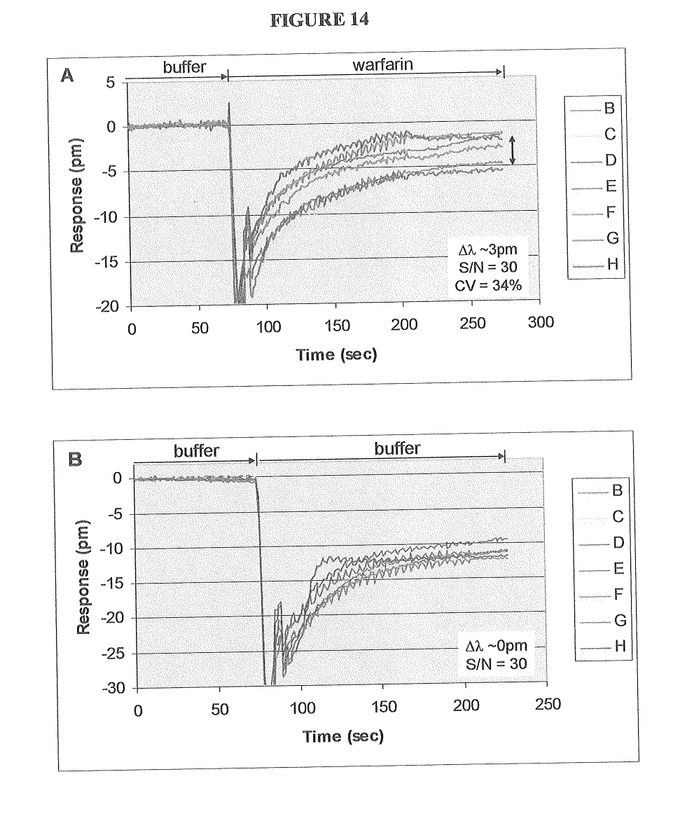

A similar set of experiments was performed using the drug warfarin. As shown in FIG. 14A, a response of ˜3 pm was observed in the four positive control wells (E-H), relative to the three negative control wells (B-D). In a control experiment, a buffer blank was introduced into each well instead of a solution of warfarin. As shown in FIG. 14B, no significant shift in response was observed between the positive and negative control wells.

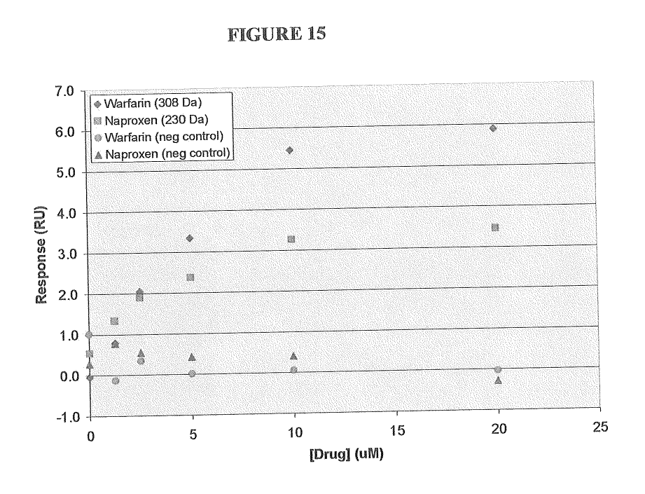

In a final demonstration, the binding profiles of the drugs warfarin (308 Da) and naproxen (230 Da) to human serum albumin immobilized on EMA were measured using SPR detection. These experiments were carried out on gold chips coated with EMA and were performed in a manner similar to that described elsewhere (14). FIG. 15 is a plot of the drug binding signal as a function of drug concentration and shows that the binding of both drugs is dose dependent. The specificity of the interaction is good as evidenced by the negligible amounts of drug binding to the negative control EMA surfaces containing no human serum albumin Note that the binding of warfarin gives rise to higher signal levels relative to naproxen, consistent with the fact that warfarin has a higher molecular weight and slightly higher affinity relative to naproxen. Scatchard analysis of the data indicated dissociation constants of ˜8 uM and ˜3 uM for naproxen and warfarin, respectively; similar experiments performed on Biacore's CM5 surface gave dissociation constants of 7.5 uM and 4.5 uM, respectively. These data are consistent with those reported in the literature and demonstrate that accurate binding data can be obtained on surfaces coated with EMA.

### D. Use of Blocking Agents

Following covalent attachment of a protein/ligand to a surface, the blocking of residual reactive groups on the surface is an important step in the study of protein-protein and/or ligand receptor interactions. Inadequate blocking can lead to high levels of non-specific binding to the surface, making analysis of results difficult if not impossible. For example, surfaces based on active-esters (e.g. N-hydroxy succinimide esters) are commonly blocked with ethanolamine to form an amide bond, thereby producing an electrically neutral, hydrophilic surface. In contrast, the reaction of an anhydride group with an amine proceeds via a ring-opening mechanism in which both an amide bond and a carboxylic acid are formed, yielding a negatively charged surface (at pH>˜4). As a result, blocking with ethanolamine or similar reagents may be insufficient for some assays and assay conditions. The novel use of electrostatic blocking agents for anhydride modified surfaces has been developed. Specifically, diethylaminethyl (DEAE) dextran is particularly effective at reducing the nonspecific binding of proteins to surfaces modified with poly(maleic anhydride-alt-methyl vinyl ether).

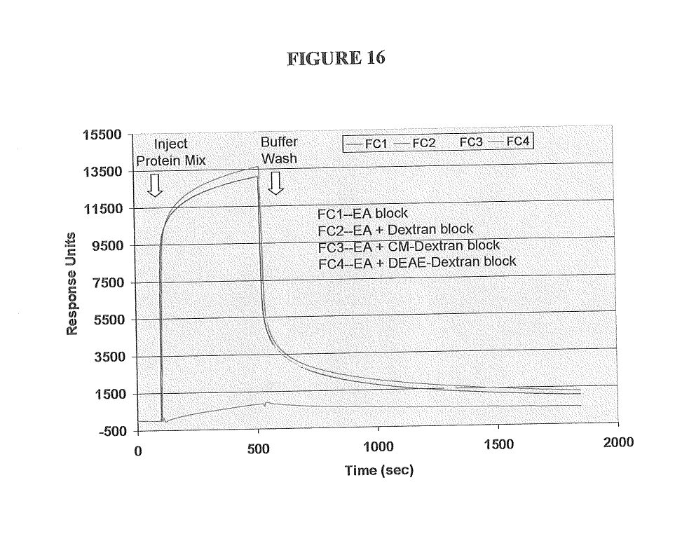

To demonstrate the use of DEAE dextran as an electrostatic blocking agent, chemically modified gold surfaces were prepared containing a thin (˜1.5 nm) layer of poly(maleic anhydride-alt-methyl vinyl ether) attached to a self-assembled monolayer of 11-mercaptoundecylamine (MUAM). After being docked into the Biacore 2000 surface plasmon resonance (SPR) instrument and equilibrated with buffer, these surfaces were reacted with ethanolamine, and then blocked for 2 minutes with either i) ethanolamine; ii) DEAE dextran, a positively charged dextran; carboxymethyl dextran, a negatively charged dextran; or iv) native dextran, which is uncharged. The amount of protein which bound to each surface was determined by injecting a solution of protein (0.5 mg/mL each of fibrinogen, lysozyme, concanavalin A, and bovine serum albumin in phosphate buffered saline, pH 7.4) over the surface for 7 minutes. (For the Biacore instrument, 1000RU corresponds to ˜1 ng/mm̂2 of adsorbed protein) Following this injection, the system was returned to buffer and washed for 2-20 minutes. FIG. 16 shows the results of this experiment. Notice that the surface blocked with ethanolamine only binds a significant amount of protein. In contrast, the surface blocked with DEAE-dextran shows substantially less binding. Specifically, after a 2 minute buffer wash, the surface blocked with ethanolamine bound ˜3.1 ng/mm̂2 (3,100 RU) of protein whereas the DEAE-dextran blocked surface bound only ˜0.74 (740 RU) of protein. Similar amounts of protein bound to surfaces blocked with either carboxymethyl dextran or native dextran, suggesting that these dextrans do not bind to the surface and that the interaction between the polymer surface and DEAE dextran is electrostatic. Nonspecifically bound protein could be removed by exposure of the surface to a solution of ethanolamine (200 mM in 150 mM borate buffer, pH9) for 5 minutes. While this wash step is effective, its use may not be compatible with low binding affinity interactions; thus, the prevention of nonspecific binding in the first place (as opposed to the removal of nonspecifically bound specifies after the fact) would be preferred.

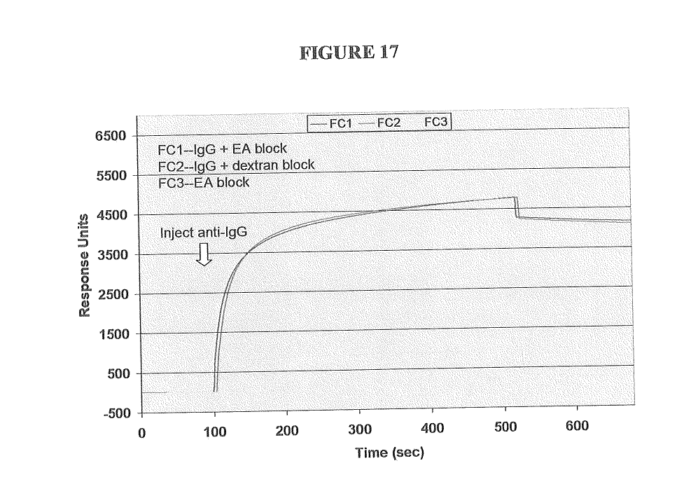

One concern with the use of a polymeric blocking agent such as DEAE-dextran is the possibility that it might interfere with the ability of analytes to bind to immobilized targets. To address this question, an SPR experiment was performed in which human IgG was immobilized on a poly(tri(ethylene glycol methyl vinyl ether)-alt-maleic anhydride) modified gold surface. Following this immobilization, flow channel 1 (FC1) was blocked with EA and flow channel 2 (FC2) was blocked with EA+DEAE dextran. Both channels were then injected with a solution of anti-IgG. As can be seen in FIG. 17, similar amounts of anti-IgG bound to both channels indicating that DEAE dextran does not interfere with IgG/anti-IgG binding.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

### REFERENCES

(1) “Immobilization of Protein Ligands with Methyl Vinyl Ether Maleic-Anhydride Copolymer,” Journal of Chromatography, 1992, 597, 123-128.
(2) “Immobilization of Saccharides and Peptides on 96-Well Microtiter Plates Coated with Methyl Vinyl Ether-Maleic Anhydride Copolymer,” Anal. Biochem., 1998, 260, 96-102.
(3) “Surface Modification via Reactive Polymer Interlayers,” Langmuir 1996, 12, 2514-2518.
(4) “Thin Polymer Layers as Supports for Hippocampal Cell Cultures,” Langmuir, 1998, 14, 3030-3035.
(5) “Surface Esterification of Poly(Ethylene-alt-Maleic Anhydride) Copolymer,” J. Phys. Chem. B, 2000, 104, 10608-10611.
(6) “Solventless Attachment of Long-Chain Molecules to Poly(ethylene-alt-maleic anhydride) Copolymer Surfaces,” J. Phys. Chem. B, 1998, 102, 5500-5502.
(7) “Oligonucleotide Synthesis on Maleic Anhydride Copolymers Covalently Bound to Silica Spherical Support and Characterization of the Obtained Conjugates,” Journal of Applied Polymer Science, 1998, 70, 2487-2497.
(8) “Oligonucleotide-Polymer Conjugates: Effect of the Method of Synthesis on Their Structure and Performance in Diagnostic Assays,” Bioconjugate Chemistry, 2000, 11, 795-804.
(9) “Covalent Immobilization of Biological Molecules to Maleic Anhydride and Methyl Vinyl Ether Copolymers-A Physico-Chemical Approach,” Journal of Applied Polymer Science, 1999, 71, 927-936.
(10) “Studies on Cyclic Anhydrides. IV. Rate Constants for the Hydrolysis of Some Cyclic Anhydrides Exhibiting Ring Strain,” Acta Chem. Scand., 1972, 26, 16-26.
(11) “Covalent Immobilization of Proteins onto (Maleic Anhydride-alt-methyl Vinyl Ether) Copolymers: Enhanced Immobilization of Recombinant Proteins,” Bioconjugate Chemistry, 1998, 9, 655-661.
(12) “Using Surface Plasmon Resonance to Study the Binding of Vancomycin and Its Dimer to Sel-Assembled Monolayers Presenting D-Ala-D-Ala,” J. Am. Chem. Soc. 1999, 121, 2629-2630.
(13) “A Survey of Structure-Property Relationships of Surfaces that Resist the Adsorption of Protein,” Langmuir, 2001, 17, 5605-5620.
(14) “Biosensor Analysis of the Interaction between Immobilized Human Serum Albumin and Drug Compounds for Prediction of Human Serum Albumin Binding Levels,” J. Med. Chem. 2000, 43, 1986-1992.
(15) “Electrostatically Driven Immobilization of Peptides onto (Maleic Anhydride-alt-methyl Vinyl Ether) Copolymers in Aqueous Media,” Ladaviere et al., Bioconjugate Chem., 2000, 11, 1460-152.
(16) “Reactive Polymers in Diagnostics: Synthesis and Characterizations of Nucleic Acid Probes and Maleic Anhydride-co-Methyl Vinyl Ether Polymers,” Ladaviere et al. J. Appl. Polymer Sci. 1997, 65, 2567-2577.
(17) “Comparison of Methods for Immobilization to Enzyme-Linked Immunosorbent Assay Plates for the Detection of Sugar Chains,” Satoh et al., Anal. Biochem., 1999, 275, 231-235.
(18) “Controlled Monomolecular Functionalization and Adhesion of Solid Surfaces,” Evenson et al., Chem Mater. 2000, 12, 3038-3043.
(19) “Pellicular Immobilized Enzymes,” Horvath, Biochim. et Biophys. Acta, 1974, 358, 164-177.
(20) International Publication No. WO8706702
(21) Japanese Publication No. JP10234847
(22) U.S. Pat. No. 4,407,975
(23) U.S. Pat. No. 4,610,962
(24) U.S. Pat. No. 4,610,962
(25) U.S. Pat. No. 4,815,843
(26) U.S. Pat. No. 6,541,071
(27) U.S. Pat. No. 6,632,615
(28) U.S. Publication No. US20030017464 A1
(29) U.S. Publication No. US20040091602 A1
(30) U.S. Publication No. US20040043497 A1
(31) U.S. Publication No. US20040043508 A1
(32) U.S. Pat. No. 5,436,161
(33) “Immobilization of Proteins to a Carboxymethyldextran-Modified Gold Surface for Biospecific Interaction Analysis in Surface Plasmon Resonance Sensors, ” Johnsson et al, Anal. Biochem., 1991, 198, 268-277.
(34) “A Novel Hydrogel Matrix on Gold Surfaces in Surface Plasmon Resonance Sensors for Fast and Efficient Covalent Immobilization of Ligands,” Lofas and Johnsson, J. Chem. Soc. Chem. Commun., 1990, 1526-1528.

1.-55. (canceled)

56. A method for preparing a label-independent surface modified microplate comprising:
attaching a first tie layer compound to the surface of the microplate;
attaching a first polymer to the first tie layer compound, the first polymer comprises a copolymer of maleic anhydride and a first monomer, the maleic anhydride in the first polymer comprises from about 5% to about 50 mol % of the first monomer; and
attaching a biomolecule to the first polymer, and the attaching is accomplished without a chemical activation step.

57. The method of claim 56, wherein the support comprises a plastic, a polymer or co-polymer substance, a ceramic, a glass, a metal, a crystalline material, a noble or semi-noble metal, a metallic or non-metallic oxide, a transition metal, or any combination thereof.

58. The method of claim 56, wherein the first tie layer is covalently attached to the outer surface of the support.

59. The method of claim 56, wherein the first tie layer is electrostatically attached to the outer surface of the support.

60. The method of claim 56, wherein the first tie-layer compound comprises one or more reactive functional groups.

61. The method of claim 60, wherein the functional group comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof.

62. The method of claim 56, wherein the first tie layer compound comprises a straight or branched-chain aminosilane, aminoalkoxysilane, aminoalkylsilane, aminoarylsilane, aminoaryloxysilane, or a derivative or salt thereof.

63. The method of claim 56, wherein the first tie layer compound comprises 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoane.

64. The method of claim 56, wherein the first tie layer is derived from a polyamine.

65. The method of claim 64, wherein the first tie layer is derived from poly-lysine or polyethyleneimine.

66. The method of claim 56, wherein the first tie layer compound comprises a self-assembled monolayer.

67. The method of claim 56, wherein the first polymer is covalently attached to the tie layer.

68. The method of claim 56, wherein the first polymer is electrostatically attached to the tie layer.

69. (canceled)

70. The method of claim 56, wherein the first polymer comprises at least one electrophilic group susceptible to nucleophilic attack.

71. The method of claim 56, wherein the first polymer comprises at least one amine-reactive group.

72. The method of claim 71, wherein the amine-reactive group comprises an ester group, an epoxide group, or an aldehyde group.

73. The method of claim 71, wherein the amine-reactive group is an anhydride group.

74. (canceled)

75. (canceled)

76. The method of claim 56, wherein the amount of maleic anhydride is about 50% by stoichiometry of the first monomer.

77. The method of claim 56, wherein the first monomer comprises styrene, tetradecene, octadecene, methyl vinyl ether, triethylene glycol methyl vinyl ether, butylvinyl ether, divinylbenzene, ethylene, acrylamide, dimethylacrylamide, pyrolidone, a polymerizable oligo(ethylene glycol) or oligo(ethylene oxide), or a combination thereof.

78. The method of claim 56, wherein the first polymer comprises, poly(vinyl acetate-maleic anhydride), poly(styrene-co-maleic anhydride),poly(ethylene-alt-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(trithyleneglycol methyvinyl ether-co-maleic anhydride), or a combination thereof.

79. The method of claim 56, wherein the first polymer is poly(ethylene-alt-maleic anhydride).

80. The method of claim 56, wherein the first polymer comprises at least one monolayer.

81. The method of claim 56, wherein the first polymer has a thickness of about 10 Å to about 2,000 Å.

82. The method of claim 56, wherein after step (2), (3) attaching a second tie layer compound to the first polymer, and (4) attaching a second polymer to the second tie layer compound.

83. The method of claim 82, wherein the second tie layer compound is covalently and/or electrostatically attached to the first polymer, and the second polymer is covalently and/or electrostatically attached to the second tie layer compound.

84. The method of claim 82, wherein the second tie layer compound comprises a polyamine or polyol.

85. The method of claim 82, wherein the second tie layer compound comprises ethylene diamine, ethylene glycol, or an oligoethylene glycol diamine.

86. The method of claim 82, wherein the second tie layer compound comprises a diamine, a triamine, or a tetraamine.

87. The method of claim 82, wherein the second polymer comprises at least one anhydride group.

88. The method of claim 82, wherein the second polymer comprises polymaleic anhydride or a copolymer derived from maleic anhydride.

89. The method of claim 56, wherein after step (2), (3) attaching a linker to the first polymer.

90. The method of claim 89, wherein the linker comprises N-(5-amino-1-carboxypentyl)iminodiacetic acid.

91. The method of claim 56, wherein after step (2), (3) attaching a biomolecule to the first polymer.

92. The method of claim 91, wherein the biomolecule is attached to the first polymer by a chemical interaction, an electrostatic interaction, or a combination thereof.

93. The method of claim 91, wherein the biomolecule is covalently attached to the first polymer.

94. The method of claim 91, wherein the biomolecule comprises a natural or synthetic oligonucleotide, a natural or modified/blocked nucleotide/nucleoside, a nucleic acid (DNA) or (RNA), a peptide comprising natural or modified/blocked amino acid, an antibody, a hapten, a biological ligand, a protein membrane, a lipid membrane, a small molecule, or a cell.

95. The method of claim 91, wherein the biomolecule comprises a protein.

96. The method of claim 95, wherein the protein comprises a peptide, a fragment of a protein or peptide, a membrane-bound protein, or a nuclear protein.

97. The method of claim 91, wherein the biomolecule is attached to the substrate in a sufficient amount under about 1 hour.

98. The method of claim 91, wherein the biomolecule is attached to the substrate in a sufficient amount under about 0.5 hours.

99. The method of claim 91, wherein the biomolecule is attached to the first polymer at a pH of from about 0.5 to 1 pH units below the isoelectric point of the biomolecule.

100. The method of claim 56, wherein after step (2), (3) attaching a blocking agent to the first polymer.

101. The method of claim 100, wherein the blocking agent comprises a positively charged compound.

102. The method of claim 100, wherein the blocking agent comprises a positively charged dextran.

103. The method of claim 100, wherein the blocking agent is diethylaminethyl dextran.

104. The method of claim 91, wherein after step (3), (4) attaching a blocking agent to the first polymer.

105. The method of claim 56, wherein the first tie layer is aminopropylsilsesquioxane and the first polymer is poly(ethylene-alt-maleic anhydride).

106. The method of claim 56, wherein the substrate is a microplate or a slide.

107. An article produced by the method of claim 56.

108. A method for performing a label-independent assay of a ligand, comprising
contacting the ligand with a substrate comprising a first tie layer, a first polymer, the first polymer comprises a copolymer of maleic anhydride and a first monomer, the maleic anhydride in the first polymer comprises from about 5% to about 50 mol % of the first monomer and a biomolecule, wherein the tie layer attaches the first polymer to the substrate, the biomolecule is attached to the first polymer without a chemical activation step, and the ligand is bound to the biomolecules on the substrate after the contacting step; and
detecting the bound ligand.

109. The method of claim 108, wherein the assay is a high-throughput assay.

110. The method of claim 108, wherein the ligand comprises a drug, an oligonucleotide, a nucleic acid, a protein, a peptide, an antibody, an antigen, a hapten, or a small molecule.

111. The method of claim 108, wherein the bound ligand is detected by fluorescence.

112. The method of claim 108, wherein the bound ligand is detected by surface plasmon resonance, a waveguide resonant grating system, or mass spectrometry.

113. The method of claim 108, wherein the substrate comprises a plastic, a polymer or co-polymer substance, a ceramic, a glass, a metal, a crystalline material, a noble or semi-noble metal, a metallic or non-metallic oxide, a transition metal, or any combination thereof.

114. The method of claim 108, wherein the first tie layer is covalently attached to the outer surface of the substrate.

115. The method of claim 108, wherein the first tie layer is electrostatically attached to the outer surface of the substrate.

116. The method of claim 108, wherein the first tie-layer compound comprises one or more reactive functional groups.

117. The method of claim 116, wherein the functional group comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof.

118. The method of claim 108, wherein the first tie layer compound comprises a straight or branched-chain aminosilane, aminoalkoxysilane, aminoalkylsilane, aminoarylsilane, aminoaryloxysilane, or a derivative or salt thereof.

119. The method of claim 108, wherein the first tie layer compound comprises 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoane.

120. The method of claim 108, wherein the first tie layer is derived from a polyamine.

121. The method of claim 120, wherein the first tie layer is derived from poly-lysine or polyethyleneimine.

122. The method of claim 108, wherein the first tie layer compound comprises a self-assembled monolayer.

123. The method of claim 108, wherein the first polymer is covalently attached to the tie layer.

124. The method of claim 108, wherein the first polymer is electrostatically attached to the tie layer.

125. The method of claim 108, wherein the first polymer comprises a copolymer.

126. The method of claim 108, wherein the first polymer comprises at least one electrophilic group susceptible to nucleophilic attack.

127. The method of claim 108, wherein the first polymer comprises at least one amine-reactive group.

128. The method of claim 127, wherein the amine-reactive group comprises an ester group, an epoxide group, or an aldehyde group.

129. The method of claim 128, wherein the amine-reactive group is an anhydride group.

130. (canceled)

131. (canceled)

132. The method of claim 130, wherein the amount of maleic anhydride is about 50% by stoichiometry of the first monomer.

133. The method of claim 130, wherein the first monomer comprises styrene, tetradecene, octadecene, methyl vinyl ether, triethylene glycol methyl vinyl ether, butylvinyl ether, divinylbenzene, ethylene, acrylamide, dimethylacrylamide, pyrolidone, a polymerizable oligo(ethylene glycol) or oligo(ethylene oxide), or a combination thereof.

134. The method of claim 108, wherein the first polymer comprises, poly(vinyl acetate-maleic anhydride), poly(styrene-co-maleic anhydride), poly(ethylene-alt-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(trithyleneglycol methyvinyl ether-co-maleic anhydride), or a combination thereof.

135. The method of claim 108, wherein the first polymer is poly(ethylene-alt-maleic anhydride).

136. The method of claim 108, wherein the first polymer comprises at least one monolayer.

137. The method of claim 108, wherein the first polymer has a thickness of about 10 Å to about 2,000 Å.

138. The method of claim 108, wherein a second tie layer compound is attached to the first polymer, and a second polymer is attached to the second tie layer compound.

139. The method of claim 138, wherein a second tie layer compound is covalently and/or electrostatically attached to the first polymer, and a second polymer is covalently and/or electrostatically attached to the second tie layer compound.

140. The method of claim 138, wherein the second tie layer compound comprises a polyamine or polyol.

141. The method of claim 138, wherein the second tie layer compound comprises ethylene diamine, ethylene glycol, or an oligoethylene glycol diamine.

142. The method of claim 138, wherein the second tie layer compound comprises a diamine, a triamine, or a tetraamine.

143. The method of claim 138, wherein the second polymer comprises at least one anhydride group.

144. The method of claim 138, wherein the second polymer comprises polymaleic anhydride or a copolymer derived from maleic anhydride.

145. The method of claim 108, wherein the substrate further comprises a linker attached to the first polymer.

146. The method of claim 145, wherein the linker comprises N-(5-amino-1-carboxypentyl)iminodiacetic acid.

147. The method of claim 108, wherein the biomolecule is attached to the first polymer by a chemical interaction, an electrostatic interaction, or a combination thereof.

148. The method of claim 108, wherein the biomolecule is attached to the first polymer by a linker.

149. The method of claim 108, wherein the biomolecule is covalently attached to the first polymer.

150. The method of claim 108, wherein the biomolecule comprises a natural or synthetic oligonucleotide, a natural or modified/blocked nucleotide/nucleoside, a nucleic acid (DNA) or (RNA), a peptide comprising natural or modified/blocked amino acid, an antibody, a hapten, a biological ligand, a protein membrane, a lipid membrane, a small molecule, or a cell.

151. The method of claim 108, wherein the biomolecule comprises a protein.

152. The method of claim 151, wherein the protein comprises a peptide, a fragment of a protein or peptide, a membrane-bound protein, or a nuclear protein.

153. The method of claim 108, wherein the substrate further comprises a blocking agent attached to the first polymer.

154. The method of claim 153, wherein the blocking agent comprises a positively charged compound.

155. The method of claim 153, wherein the blocking agent comprises a positively charged dextran.

156. The method of claim 153, wherein the blocking agent is diethylaminethyl dextran.

157. The method of claim 108, wherein the first tie layer is aminopropylsilsesquioxane and the first polymer is poly(ethylene-alt-maleic anhydride).

158. The method of claim 108, wherein the substrate is a microplate or a slide.

159. The article of claim 1, wherein the substrate is a microplate, the first tie layer is derived from 3-aminopropyl trimethoxysilane, and the first polymer is poly(ethylene-alt-maleic anhydride).

160. The method of claim 108, wherein the bound ligand is detected by label independent detection.

161. The method of claim 108, wherein the substrate is a microplate, the first tie layer is derived from 3-aminopropyl trimethoxysilane, the first polymer is poly(ethylene-alt-maleic anhydride), and the bound ligand is detected by label independent detection.
